(19) United States
(12) Patent Application Publication
(10) Pub. No.: US 2019/0209229 A1
(43) Pub. Date: Jul. 11, 2019

(54) DEVICE FOR MONITORING TEMPERATURES WITHIN AND ADJACENT TO BODY LUMENS

(71) Applicant: Adagio Medical, Inc., Laguna Hills, CA (US)

(72) Inventors: Alexei Babkin, Dana Point, CA (US); Zachary Zira, Laguna Hills, CA (US)

(21) Appl. No.: 16/243,451

(22) Filed: Jan. 9, 2019

Related U.S. Application Data
(60) Provisional application No. 62/615,807, filed on Jan. 10, 2018.

(51) Int. Cl. A61B 18/02; A61B 5/01; A61B 5/00

(52) U.S. Cl. CPC A61B 18/02; A61B 2018/00815; A61B 5/6853; A61B 5/01

(57) ABSTRACT

A device for monitoring temperature adjacent to an ablation site, the device having an expandable component and a shaft that includes a lumen, at least one fluid port in communication with the lumen, and a plurality of flexible elements having a first end connected to the shaft and a second end having a temperature sensor mounted thereon.

### CROSS-REFERENCE TO RELATED APPLICATIONS

This application claims the benefit of U.S. Provisional Application No. 62/615,807, filed Jan. 10, 2018, the entire contents of which are incorporated herein by reference in their entirety for all purposes.

### BACKGROUND

### 1. Field of the Invention

Embodiments of the invention generally relate to expandable temperature monitoring and controlling devices and, more particularly, to a balloon for use in ablation procedures to warm/cool and/or monitor temperatures adjacent to the location of the ablation treatment.

### 2. Description of the Related Art

One of the more prevalent types of heart disease or conditions is atrial fibrillation (AF). Atrial fibrillation is an irregular and often rapid heart rate. The heart's electrical signals fail to travel normally, and spread throughout the atria of the heart in a rapid, disorganized way. Failing to treat atrial fibrillation can lead to a number of undesirable consequences including heart palpitations, shortness of breath, weakness and generally poor blood flow to the body.

Various techniques are practiced to treat atrial fibrillation. One technique to treat AF is pulmonary vein isolation (PVI). PVI is performed by creating lesions circumscribing the pulmonary veins. The PVI serves to block the errant or abnormal electrical signals.

Different types of thermal ablation such as, for example, cryoablation, radiofrequency ablation, microwave ablation, laser ablation, and high frequency ultrasound (HIFU) can be used to create the lesions for PVI. However, for these lesions to be effective in treating AF, the thermal ablation used must create transmural lesions through the entire heart wall thickness to effectively eliminate flow of the errant electrical signals within the heart walls. Creating these lesions through the entire wall thickness can be problematic and dangerous as damage to surrounding anatomy must be avoided.

As can be seen in FIG. 1, the pulmonary vein entries 2 in the left atrium of the heart 4 are in very close proximity to the esophagus 6. Thus, when forming lesion(s) in the vicinity of these pulmonary vein entries 2, the potential exists for the lesion to extend through the heart wall and to continue into the esophagus 6 thereby potentially creating an esophageal fistula. Typically, esophageal fistulas and the associated complications do not occur immediately after the ablation procedure and when they finally present themselves, which can be weeks later, it is usually too late to treat and can be fatal.

In an effort to try to avoid damaging the esophagus during an ablation procedure to treat conditions in the heart, AF for example, temperature sensing devices and heating or cooling devices, depending on the type of ablation being performed, have been placed in the esophagus in order to monitor the temperature in and/or heat/cool the esophageal tissue adjacent to the ablation site within the heart. Prior devices used for temperature monitoring include catheters and probes that comprise temperature sensors thereon.

An example of a prior temperature sensing device is described in U.S. Pat. No. 9,155,476. The device described in the '476 patent comprises a flexible temperature probe that includes a plurality of temperature sensors on its distal portion. In use, the distal portion is inserted into the esophagus and maneuvered such that the temperature sensors are positioned against the internal surface of the esophagus for measuring the internal surface temperature of the esophagus. While it is important to know the temperature in the esophagus, just knowing the temperature may not be sufficient as a person's anatomy may make it difficult to prevent tissue damage and hence, lesion formation and may require discontinuing the ablation procedure. Further, the device in the '476 patent includes discreet temperature sensors that measure temperature at discreet points along the distal portion. Therefore, the device does not measure tissue temperature between the temperature sensors.

Another example of a prior device used to heat or cool the area of the esophagus adjacent to the ablation site within the heart include inflatable balloons. In use, the inflatable balloon is positioned in the esophagus adjacent the ablation site and either a heating fluid or cooling fluid (depending on whether cooling ablation or heating ablation is performed), is circulated through the balloon, in an attempt to maintain the tissue of the esophagus at a temperature that will not damage the tissue. Even though circulating a heating or cooling fluid through the balloon may increase or reduce the temperature of the esophageal tissue, damage to the esophagus may still occur as the temperature of the tissue is not known, which may still cause the tissue to reach damaging temperatures.

Accordingly, as a result of the above-described shortcomings of prior devices, there is a need for an improved device that can accurately monitor temperature in a body lumen or cavity such as, for example, the esophagus, while also warming or cooling the lumen or body cavity tissue.

### SUMMARY

Some embodiments of the invention are directed to a device for monitoring temperature adjacent to an ablation site, the device having an expandable component and a shaft that includes a lumen, at least one fluid port in communication with the lumen, and a plurality of flexible elements having a first end connected to the shaft and a second end having a temperature sensor mounted thereon. In some embodiments of the invention, the temperature sensors are mounted intermediate the first and second ends.

Embodiments of the invention are also directed to a device for monitoring temperature adjacent to an ablation site where the device comprises an expandable component and a shaft. The shaft includes a lumen, at least one fluid port in communication with the lumen, a plurality of flexible elements having a first end connected to the shaft and a second end having a temperature sensor mounted thereon and a paddle structure.

Embodiments of the invention are also directed to a method of monitoring temperature in a body lumen where the method comprises delivering a device to a site of interest in the lumen, the device having an expandable component, at least one temperature sensor and at least one fluid port. The method also includes filling the expandable component with a fluid and detecting a temperature of the fluid with the temperature sensor.

Embodiments of the invention are also directed to a method of monitoring tissue temperature within a human body where the method comprises delivering a device to a site of interest in the human body, the device having a balloon, at least one temperature sensor on an interior of the balloon and at least one fluid port to supply a fluid to the interior of the balloon. The method also includes filling the balloon with the fluid and detecting a temperature of the fluid with the temperature sensor.

Embodiments of the invention are also directed to a method of monitoring tissue temperature within a human body where the method comprises delivering a device to a site of interest in the human body. The device includes a balloon, at least one temperature sensor on an interior of the balloon and at least one fluid port to supply a fluid to the interior of the balloon. The method also includes filling the balloon with the fluid, stirring the fluid within the balloon and detecting a temperature of the fluid with the temperature sensor.

Embodiments of the invention are also directed to a method of performing a cryoablation procedure comprising delivering a cryoablation catheter to a site of interest within a human body, delivering a tissue warming device to a location within the human body adjacent to the site of interest, commencing flow of a warming fluid to the tissue warming device, stirring the warming fluid in the tissue warming device, monitoring a temperature if the stirred fluid within the tissue warming device and performing cryoablation at the site of interest while monitoring the temperature of the stirred fluid within the tissue warming device.

Embodiments of the invention are also directed to a method of performing an ablation procedure that uses heat to ablate tissue where the ablation procedure comprises delivering an ablation energy device to a site of interest within a human body, delivering a tissue cooling device to a location within the human body adjacent to the site of interest, commencing flow of a cooling fluid to the tissue cooling device, stirring the cooling fluid in the tissue cooling device, monitoring a temperature of the stirred fluid within the tissue cooling device and ablating tissue at the site of interest while monitoring the temperature of the stirred fluid within the tissue cooling device.

Embodiments of the invention are also directed to a method of performing a cryoablation procedure comprising delivering a cryoablation energy device to a site of interest within a human body and delivering a tissue warming device to a location within the human body adjacent to the site of interest. In some embodiments, the tissue warming device includes a balloon, at least one temperature sensor on an interior of the balloon and at least one fluid port to supply warming fluid to the interior of the balloon. The method also includes filling the balloon with a warming fluid, monitoring a temperature of the warming fluid within the tissue warming device and performing cryoablation at the site of interest while monitoring the temperature of the warming fluid within the balloon.

Embodiments of the invention are also directed to a device for monitoring temperature adjacent to an ablation site where the device comprises an expandable component and a shaft having a lumen, at least one fluid port in communication with the lumen and at least one paddle structure having at least one temperature sensor mounted thereon.

In some embodiments, the invention is directed to a method of performing a cryoablation procedure comprising delivering a cryoablation energy device to a site of interest within a human body, delivering a tissue warming device to a location within the human body adjacent to the site of interest where the tissue warming device includes a balloon, at least one temperature sensor on an interior of the balloon and at least one fluid port to supply warming fluid to the interior of the balloon. The method also includes filling the balloon with a warming fluid, monitoring a temperature of the warming fluid within the tissue warming device and performing cryoablation at the site of interest while monitoring the temperature of the warming fluid within the balloon.

Embodiments of the invention are also directed to a method of performing a cryoablation procedure comprising delivering a cryoablation energy device to a site of interest within a human body, delivering a tissue warming device to a location within the human body adjacent to the site of interest where the tissue warming device includes a balloon, at least one temperature sensor on an interior of the balloon and at least one fluid port to supply warming fluid to the interior of the balloon. The method also includes filling the balloon with a warming fluid, monitoring a temperature of the warming fluid within the balloon, performing cryoablation at the site of interest while monitoring the temperature of the warming fluid within the balloon, analyzing the monitored temperature and halting the cryoablation procedure if the temperature of the warming fluid within the balloon meets or exceeds predetermined temperature parameters.

In some embodiments, the invention is directed to a method of performing a cryoablation procedure in a left atrium of the heart where the method comprises the steps of advancing a tissue warming device to a location within an esophagus adjacent to a wall of the left atrium where the ablation procedure is to be performed. In some embodiments, the tissue warming device comprises a balloon, at least one temperature sensor on an interior of the balloon and at least one fluid port to supply warming fluid to the interior of the balloon. The method further includes advancing a cryoablation catheter into the left atrium, navigating a treatment section of the cryoablation catheter to an area in the heart near a pulmonary vein entry, filling the balloon with a warming fluid, monitoring a temperature of the warming fluid within the tissue warming device and performing at least one cryoablation cycle to create a lesion near the pulmonary vein entry while monitoring the temperature of the warming fluid within the balloon.

Further embodiments of the invention are directed to a device for monitoring temperature adjacent to a body lumen. The device comprises an expandable component and a shaft. The shaft comprises a lumen, at least one fluid port in communication with the lumen, and at least one flexible paddle structure connected to the shaft where the paddle structure comprises (i) a frame structure, (ii) a film component and (iii) at least one temperature sensor mounted thereon.

Embodiments of the invention are also directed to a device for monitoring temperature adjacent to a body lumen comprising a balloon and a shaft on an interior of the balloon. In some embodiments, the shaft comprises a lumen, at least one fluid port in communication with the lumen and a plurality of flexible paddle structures connected to the shaft, where each paddle structure comprises (i) a nitinol frame structure and (ii) a plastic film component attached to the frame structure. At least one of flexible paddle structures includes at least one temperature sensor mounted thereon.

In some embodiments, the invention is directed to a method of performing an ablation procedure comprising delivering an ablation energy device to a site of interest within a human body, delivering a tissue temperature monitoring device to a location within the human body adjacent to the site of interest. In some embodiments, the tissue temperature monitoring device comprises an expandable component, at least one temperature sensor on an interior of the expandable component, and at least one fluid port to supply a fluid to the interior of the expandable component. The method also includes filling the expandable component with the fluid, monitoring a temperature of the fluid within the expandable component and performing an ablation at the site of interest while monitoring the temperature of the fluid within the expandable component.

Additional embodiments of the invention are directed to methods of performing an ablation procedure where the method comprises delivering an ablation energy device to a site of interest within a human body, delivering a tissue temperature monitoring device to a location within the human body adjacent to the site of interest, where the tissue temperature monitoring device includes an expandable component and a shaft. The shaft comprises a lumen, at least one fluid port in communication with the lumen and at least one paddle structure connected to the shaft, where the at least one paddle structure comprises (i) a frame structure, (ii) a film component and (iii) at least one temperature sensor mounted thereon. The method also includes filling the expandable component with a fluid, monitoring the temperature of the fluid within the expandable component and performing an ablation at the site of interest while monitoring the temperature of the fluid within the expandable component.

Further embodiments of the invention are directed to a method of performing a cryoablation procedure where the method comprises delivering a cryoablation energy device to a site of interest within a human body, delivering a tissue temperature monitoring device to a location within the human body adjacent to the site of interest, where the tissue temperature monitoring device comprises a balloon and a shaft. In some embodiments, the shaft comprises a lumen, at least one fluid port in communication with the lumen and at least one paddle structure connected to the shaft and comprising (i) a frame structure, (ii) a film component and (iii) at least one temperature sensor mounted thereon. The method also includes filling the balloon with a warming fluid, monitoring a temperature of the warming fluid within the balloon and performing cryoablation at the site of interest while monitoring the temperature of the warming fluid within the balloon.

Additional embodiments of the invention are directed to a device for monitoring temperature adjacent to a body lumen where the device comprises an expandable component and a first shaft comprising having a lumen, at least one fluid port in communication with the lumen, at least one paddle structure connected to the shaft and comprising (i) a frame structure and (ii) a film component, and at least one temperature sensor mounted the first shaft. The device also comprises a second shaft surrounding at least a portion of the first shaft and having a gap formed between the first shaft and the second shaft, wherein the gap is for supplying a fluid to an interior of the expandable component.

Some embodiments of the invention are directed to a device for monitoring temperature adjacent to a body lumen. The device comprises a balloon and a shaft on an interior of the balloon, where the shaft comprises a lumen at least one fluid port in communication with the lumen a plurality of flexible paddle structures connected to the shaft, where each paddle structure includes (i) a nitinol frame structure and (ii) a plastic film component attached to the frame structure, and at least one temperature sensor mounted on the shaft.

Additionally, in some embodiments, a method of performing an ablation procedure is disclosed. The method comprises delivering an ablation energy device to a site of interest within a human body, delivering a tissue temperature monitoring device to a location within the human body adjacent to the site of interest, where the tissue temperature monitoring device comprises an expandable component and a shaft comprising a lumen, at least one fluid port in communication with the lumen, at least one paddle structure connected to the shaft and having (i) a frame structure and (ii) a film component, and at least one temperature sensor mounted on the shaft. The method further includes filling the expandable component with a fluid, monitoring the temperature of the fluid within the expandable component and performing an ablation at the site of interest while monitoring the temperature of the fluid within the expandable component.

A further embodiment of the invention is directed to a method of performing a cryoablation procedure comprising delivering a cryoablation energy device to a site of interest within a human body, delivering a tissue temperature monitoring device to a location within the human body adjacent to the site of interest, the tissue temperature monitoring device comprising a balloon and a shaft having at least one fluid port, at least one paddle structure connected to the shaft and comprising (i) a frame structure and (ii) a film component, and at least one temperature sensor mounted on the shaft. The method also includes filling the balloon with a warming fluid, monitoring the temperature of the warming fluid within the balloon, and performing cryoablation at the site of interest while monitoring the temperature of the warming fluid within the balloon.

In another embodiment, the invention is directed to a method of performing an ablation procedure comprising delivering an ablation energy device to a site of interest within a human body, delivering a tissue temperature monitoring device to a location within the human body adjacent to the site of interest, where the tissue temperature monitoring device comprises an expandable component, a first shaft having a lumen, at least one fluid port in communication with the lumen, at least one paddle structure connected to the shaft and comprising (i) a frame structure and (ii) a film component, and at least one temperature sensor mounted the first shaft. The tissue temperature monitoring device also includes a second shaft surrounding at least a portion of the first shaft and having a gap formed between the first shaft and the second shaft, wherein the gap is for supplying a fluid to an interior of the expandable component. The method further includes filling the expandable component with the fluid through the gap formed between the first shaft and the second shaft, monitoring a temperature of the fluid within the expandable component and performing ablation at the site of interest while monitoring the temperature of the fluid within the expandable component.

The description, objects and advantages of embodiments of the present invention will become apparent from the detailed description to follow, together with the accompanying drawings.

### BRIEF DESCRIPTION OF THE DRAWINGS

The above-mentioned aspects, as well as other features, aspects and advantages of the present technology will now be described in connection with various embodiments, with reference to the accompanying drawings. The illustrated embodiments, however, are merely examples and are not intended to be limiting. Throughout the drawings, similar symbols typically identify similar components, unless context dictates otherwise. Note that the relative dimensions of the following FIGS. may not be drawn to scale.

FIG. 1 illustrates human anatomy in the vicinity of the heart;

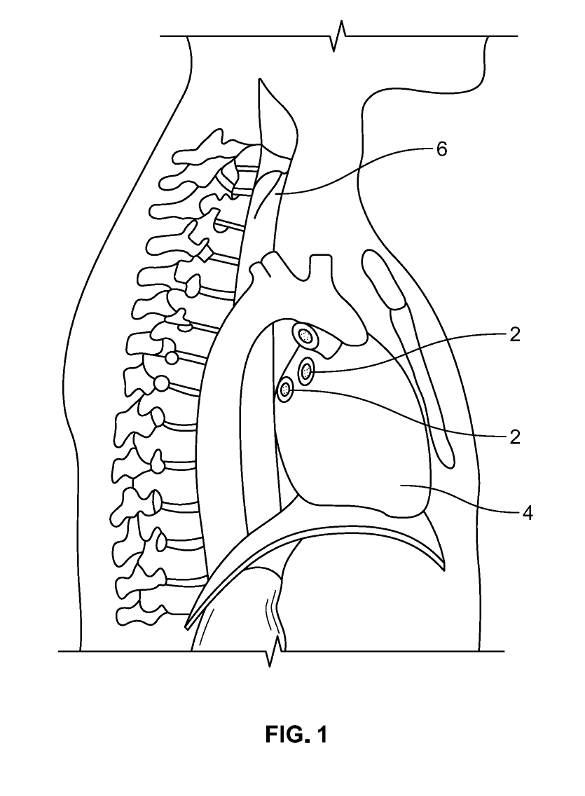

FIG. 2 depicts a device inserted into a body lumen, according to an embodiment of the invention;

FIG. 3 is a perspective view of a device, according to an embodiment of the invention;

FIG. 4 depicts the distal end of the shaft/hollow tube-like structure, according to an embodiment of the invention;

FIG. 5A depicts a device having a paddle structure inserted into a body lumen, according to an embodiment of the invention;

FIG. 5B depicts the shaft/hollow tube-like structure having two paddle structures, according to an embodiment of the invention;

FIG. 5C depicts the shaft/hollow tube-like structure having two paddle structures, according to an embodiment of the invention;

FIG. 5D depicts a device having a spiraled paddle structure, according to an embodiment of the invention;

FIG. 5E depicts a device having a plurality of paddle structures, according to an embodiment of the invention; and

FIG. 6 depicts a device inserted into a body lumen, according to an embodiment of the invention.

### DETAILED DESCRIPTION

It is to be understood that the embodiments of the invention described herein are not limited to particular variations set forth herein as various changes or modifications may be made to the embodiments of the invention described and equivalents may be substituted without departing from the spirit and scope of the embodiments of the invention. As will be apparent to those of skill in the art upon reading this disclosure, each of the individual embodiments described and illustrated herein has discrete components and features that may be readily separated from or combined with the features of any of the other several embodiments without departing from the scope or spirit of the embodiments of the present invention. In addition, many modifications may be made to adapt a particular situation, material, composition of matter, process, process act(s) or step(s) to the objective(s), spirit or scope of the embodiments of the present invention. All such modifications are intended to be within the scope of the claims made herein.

Moreover, while methods may be depicted in the drawings or described in the specification in a particular order, such methods need not be performed in the particular order shown or in sequential order, and that all methods need not be performed, to achieve desirable results. Other methods that are not depicted or described can be incorporated in the example methods and processes. For example, one or more additional methods can be performed before, after, simultaneously, or between any of the described methods. Further, the methods may be rearranged or reordered in other implementations. Also, the separation of various system components in the implementations described above should not be understood as requiring such separation in all implementations, and it should be understood that the described components and systems can generally be integrated together in a single product or packaged into multiple products. Additionally, other implementations are within the scope of this disclosure.

Conditional language, such as “can,” “could,” “might,” or “may,” unless specifically stated otherwise, or otherwise understood within the context as used, is generally intended to convey that certain embodiments include or do not include, certain features, elements, and/or steps. Thus, such conditional language is not generally intended to imply that features, elements, and/or steps are in any way required for one or more embodiments.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require the presence of at least one of X, at least one of Y, and at least one of Z.

Reference to a singular item, includes the possibility that there are plural of the same items present. More specifically, as used herein and in the appended claims, the singular forms “a,” “an,” “said” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It will be understood that when an element is referred to as being “connected” or “coupled” to another element, it can be directly connected or coupled to the other element or intervening elements may be present. In contrast, if an element is referred to as being “directly connected” or “directly coupled” to another element, there are no intervening elements present.

It will also be understood that, although the terms first, second, etc. may be used herein to describe various elements, these elements should not be limited by these terms. These terms are only used to distinguish one element from another. Thus, a first element could be termed a second element without departing from the teachings of the present invention.

Language of degree used herein, such as the terms “approximately,” “about,” “generally,” and “substantially,” represent a value, amount, or characteristic close to the stated value, amount, or characteristic that still performs a desired function or achieves a desired result. For example, the terms “approximately,” “about,” “generally,” and “substantially” may refer to an amount that is within less than or equal to 10% of, within less than or equal to 5% of, within less than or equal to 1% of, within less than or equal to 0.1% of, and within less than or equal to 0.01% of the stated amount. If the stated amount is 0 (e.g., none, having no), the above recited ranges can be specific ranges, and not within a particular % of the value. Additionally, numeric ranges are inclusive of the numbers defining the range, and any individual value provided herein can serve as an endpoint for a range that includes other individual values provided herein. For example, a set of values such as 1, 2, 3, 8, 9, and 10 is also a disclosure of a range of numbers from 1-10, from 1-8, from 3-9, and so forth.

Some embodiments have been described in connection with the accompanying drawings. Distances, angles, etc. are merely illustrative and do not necessarily bear an exact relationship to actual dimensions and layout of the devices illustrated. Components can be added, removed, and/or rearranged. Further, the disclosure herein of any particular feature, aspect, method, property, characteristic, quality, attribute, element, or the like in connection with various embodiments can be used in all other embodiments set forth herein. Additionally, it will be recognized that any methods described herein may be practiced using any device suitable for performing the recited steps.

While a number of embodiments and variations thereof have been described in detail, other modifications and methods of using the same will be apparent to those of skill in the art. Accordingly, it should be understood that various applications, modifications, materials, and substitutions can be made of equivalents without departing from the unique and inventive disclosure herein or the scope of the claims.

All existing subject matter mentioned herein (e.g., publications, patents, patent applications and hardware) is incorporated by reference herein in its entirety except insofar as the subject matter may conflict with that of the present invention (in which case what is present herein shall prevail).

Thermal ablation may be used to treat many conditions and diseases including, and not limited to, cancerous tissue and atrial fibrillation. These conditions and diseases can be treated in many organs of the human body including, and not limited to, the heart, liver, lungs, kidneys, prostate, bladder, ovaries, cervix, uterus, endometrium, breasts, brain, stomach, colon and skin. In treating certain conditions, it is imperative not to damage healthy tissue adjacent to the ablation site. Accordingly, embodiments of the present invention are directed to devices and methods that monitor temperature and maintain a safe temperature in tissue adjacent to the ablation site. In some embodiments of, the temperature of the tissue is monitored by monitoring the fluid flowing on the interior of the device.

Embodiments of the present invention can be used with various cryoablation systems, their components, and various arrangements. Examples of these cryoablation systems, their components, and various arrangements are described in the following commonly-assigned U.S. patents and U.S. patent applications: U.S. patent application Ser. No. 10/757,768, which issued as U.S. Pat. No. 7,410,484, on Aug. 12, 2008 entitled “CRYOTHERAPY PROBE,” filed Jan. 14, 2004 by Peter J. Littrup et al.; U.S. patent application Ser. No. 10/757,769, which issued as U.S. Pat. No. 7,083,612 on Aug. 1, 2006, entitled “CRYOTHERAPY SYSTEM,” filed Jan. 14, 2004 by Peter J. Littrup et al.; U.S. patent application Ser. No. 10/952,531, which issued as U.S. Pat. No. 7,273,479 on Sep. 25, 2007 entitled “METHODS AND SYSTEMS FOR CRYOGENIC COOLING,” filed Sep. 27, 2004 by Peter J. Littrup et al.; U.S. patent application Ser. No. 11/447,356, which issued as U.S. Pat. No. 7,507,233 on Mar. 24, 2009 entitled “CRYOTHERAPY SYSTEM,” filed Jun. 6, 2006 by Peter Littrup et al.; U.S. patent application Ser. No. 11/846,226, which issued as U.S. Pat. No. 7,921,657 on Apr. 12, 2011 entitled “METHODS AND SYSTEMS FOR CRYOGENIC COOLING,” filed Aug. 28, 2007 by Peter Littrup et al.; U.S. patent application Ser. No. 12/018,403, which issued as U.S. Pat. No. 8,591,503 on Nov. 26, 2013 entitled “CRYOTHERAPY PROBE,” filed Jan. 23, 2008 by Peter Littrup et al.; U.S. patent application Ser. No. 13/046,274, which issued as U.S. Pat. No. 8,387,402 on Mar. 5, 2013 entitled “METHODS AND SYSTEMS FOR CRYOGENIC COOLING,” filed Mar. 11, 2011 by Peter Littrup et al.; U.S. patent application Ser. No. 14/087,947, which is pending entitled “CRYOTHERAPY PROBE,” filed Nov. 22, 2013 by Peter Littrup et al.; U.S. patent application Ser. No. 12/744,001, which issued as U.S. Pat. No. 8,740,891, on Jun. 3, 2014 entitled “FLEXIBLE MULTI-TUBULAR CRYOPROBE,” filed Jul. 29, 2010 by Alexei Babkin et al.; U.S. patent application Ser. No. 12/744,033, which issued as U.S. Pat. No. 8,740,892, on Jun. 3, 2014 entitled “EXPANDABLE MULTI-TUBULAR CRYOPROBE,” filed Jul. 29, 2010 by Alexei Babkin et al. and U.S. patent application Ser. No. 14/915,632 entitled “ENDOVASCULAR NEAR CRITICAL FLUID BASED CRYOABLATION CATHETER AND RELATED METHODS,” filed Sep. 22, 2014 by Alexei Babkin, et al., the contents of each of the above-identified U.S. patents/applications are incorporated herein by reference in their entireties for all purposes.

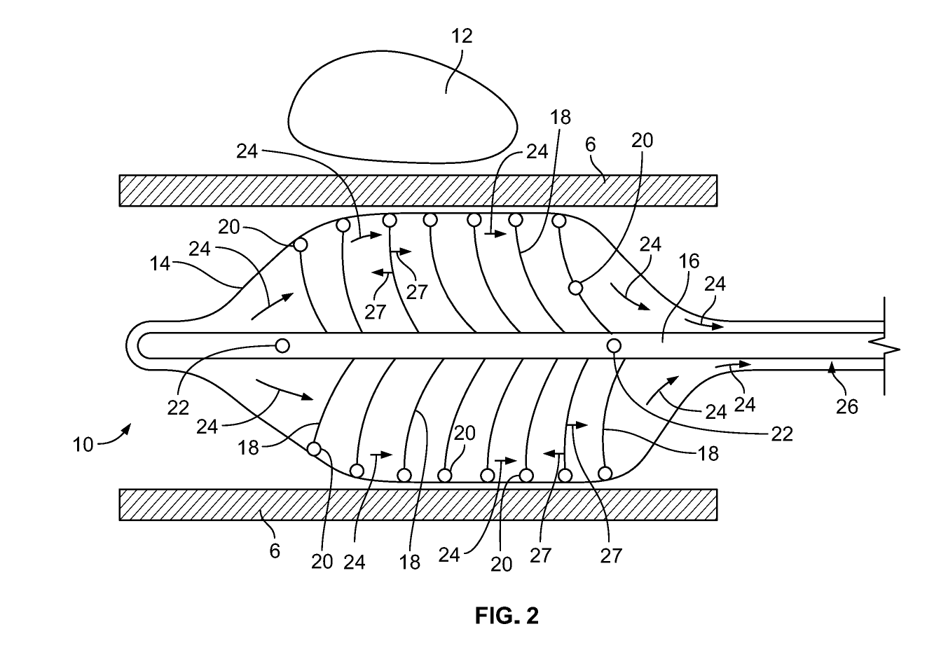

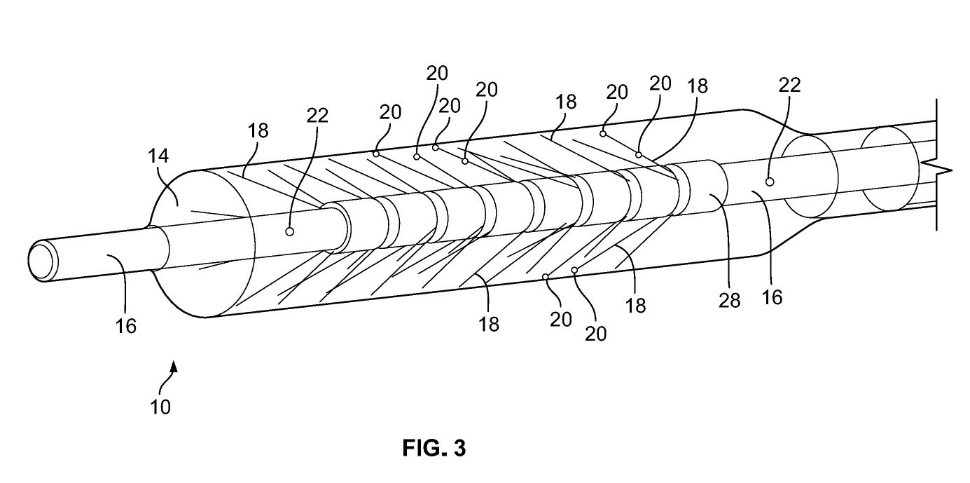

Depicted in FIGS. 2 and 3 is device according to an embodiment of the present invention. As shown, the device 10 is inserted into a body lumen 6 adjacent to an ablation site 12. In the disclosed and described embodiments, the device 10 is inserted into the esophagus 6 and positioned adjacent to the ablation site 12. Use of the device, however, is not limited to the esophagus and can be used in any body lumen such as, for example, the small intestine, colon, rectum, vascular system, renal system, etc. In the disclosed and described embodiments, the ablation being performed is cryoablation, however, as previously discussed, embodiments of the invention can be used with other types of ablation including, and not limited to, radiofrequency ablation, microwave ablation, laser ablation, and high frequency ultrasound (HIFU). Additionally, embodiments of the device disclosed and described herein can also be used outside of body lumens and can be placed directly into tissue in the vicinity of the ablation site or into body cavities such as, for example, the abdomen, in the vicinity of the ablation site.

In this embodiment, the device 10 includes an expandable component 14 such as, for example, a balloon, and a shaft/hollow tube-like structure 16 upon which are mounted a plurality of flexible elements 18. In some embodiments, the flexible elements 18 can be nitinol wires. As will be readily understood to those skilled in the art, any flexible material may be used for the flexible elements 18.

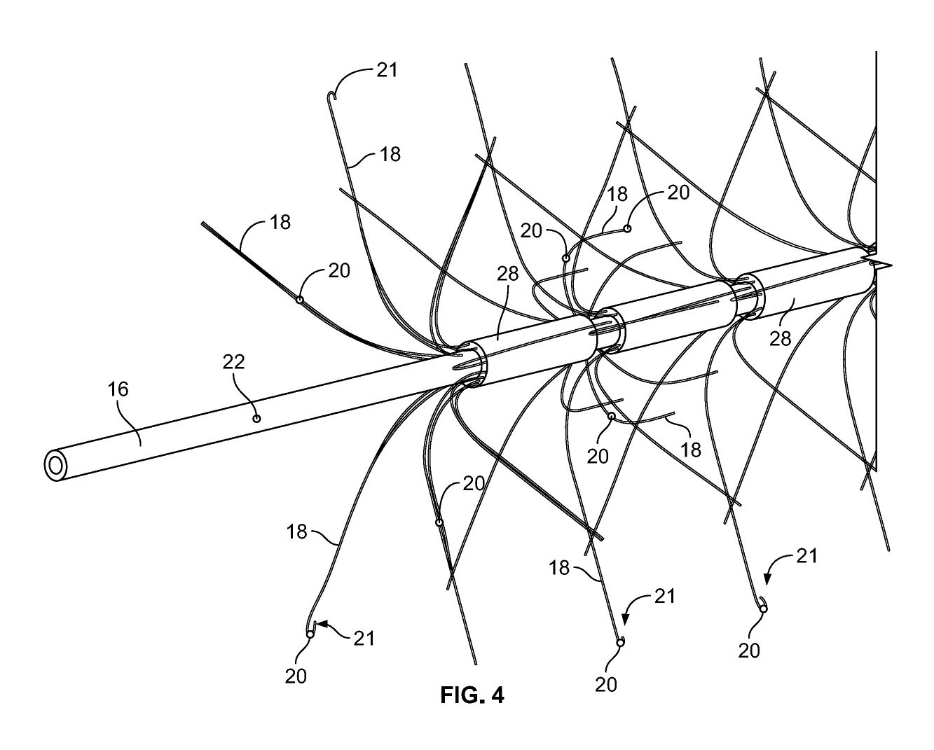

Included on the free ends of the flexible elements 18 (the ends not connected to the shaft/hollow tube-like structure 16) are temperature sensors 20, which are used to measure/monitor either (1) the temperature of the fluid (as discussed below) within the expandable component 14 or (2) the temperature of the tissue that the exterior of the expandable component 14 contacts adjacent to the temperature sensor 20. Examples of temperature sensors 20 that can be used include, and are not limited to, thermocouples, Resistance Temperature Detectors (RTDs) and thermistors. As will be understood by those of skill in the art, measuring temperature may be achieved with any other device that measures temperature. In some embodiments, the temperature sensors 20 are included on elements that prevent the flexible elements 18 from puncturing the expandable component 14. In some embodiments, the temperature sensors 20 are included on ball-like or spherical elements. As will be understood by those of skill in the art, these non-puncturing elements can be any shape or configuration so long as the shape or configuration does not puncture or otherwise damage the expandable component 14. In some embodiments, as depicted in FIG. 4, the free ends 21 of the flexible elements 18 are curved back towards the flexible elements 18 such that the free ends 21 are smooth and therefore, will not puncture or damage the expandable component 14. Temperature sensors 20 may be placed on these curved free ends 21 as these will contact the inner surface of the expandable component 14 and will therefore, measure the temperature of the tissue that the exterior surface of the expandable component 14 contacts. In some embodiments, the length of the flexible elements 18 is sufficient to ensure that when the expandable components 14 are fully expanded/spring open, the temperature sensors 20 are in sufficient contact with the inner surface of the expandable component 14.

In some embodiments, the temperature sensors 20 need not contact the inner surface of the expandable component 14. In these embodiments, the temperature sensors 20 just extend into the interior volume of the expandable component 14 and measure the temperature of the warming/cooling fluid flowing therein as discussed below.

In some embodiments, more than one temperature sensor 20 is included on a flexible element 18. Including multiple temperature sensors 20 along the length of the flexible element 18 allows greater temperature monitoring of the fluid on the interior of the expandable component 14, which is important for the reasons discussed herein.

In some embodiments, the control wires for the temperature sensors 20 are attached to or incorporated within the flexible elements 18. In some embodiments, the control wires for the temperature sensors are included within the shaft/hollow tube-like structure 16.

As can be seen in FIG. 2, the shaft/hollow tube-like structure 16 includes at least one fluid port 22, which allows fluid to be delivered from the interior of the shaft/hollow tube-like structure 16 to the interior of the expandable component 14. In some embodiments, 2, 3, 4, 5, 6 or more fluid ports 22 are included. The fluid ports 22 can be used to supply fluid or remove fluid from the interior of the expandable component 14. For cryoablation, this fluid will be a warming fluid that warms the tissue of the esophagus 6 in order to prevent damage to the tissue (i.e., to prevent the tissue from freezing). This fluid can either be warmed prior to entering the internal lumen of the shaft/hollow tube-like structure 16 on the exterior of the human body or the fluid can enter the shaft/hollow tube-like structure 16 at room temperature and can be warmed as it flows through the shaft/hollow tube-like structure 16 within the human body such that it is fully warmed by the time it exits the fluid ports 22. For warming prior to entering the shaft/hollow tube-like structure 16 on the exterior of the human body, a separate warming device such as for example, a fluid warmer may be used. Heat generated from the human body can also be used to warm the fluid. The fluid would be inserted through the device and into the expandable component at room temperature, then heat transfer from the body can warm the fluid to body temperature. In embodiments that require cooling to prevent damage to tissue adjacent the ablation site, a separate cooling device such as for example, a fluid cooler may be used. As will be understood by those of skill in the art, any device that cools the fluid may be used.

In some embodiments, the device can be used with ablation technologies that use heat to ablate tissue. These ablation technologies include, and are not limited to, microwave ablation and RF ablation. When used with ablation technologies that generate heat, the fluid will be a cooling fluid in order to prevent damage to tissue adjacent the ablation site. In these embodiments, a separate cooling device such as for example, a fluid cooler may be used. As will be understood by those of skill in the art, any device that cools the fluid may be used.

In some embodiments, the primary purpose of the device may not be to cool or heat the tissue adjacent to the ablation site. Instead, the purpose of the device may be to monitor/measure the temperature adjacent to the ablation site. In these embodiments, the fluid need not be cooled or warmed.

Examples of fluids that can be used with the embodiments of the device disclosed and described herein include, and are not limited to water and saline. As will be ready understood by those of skill in the art, any fluid that can be used as a cooling fluid, warming fluid or fluid that best transfers heat/cold for better temperature sensing, may be used. In some embodiments, a radiopaque material such as, for example, contrast is included/added to the fluid in order to help visualize the device in the body with visualization technologies such as, for example, fluoroscopy.

After the warming/cooling fluid exits the fluid ports 22, it flows around the interior of the expandable component 14 as depicted by arrows 24 in FIG. 2. In some embodiments, the direction of fluid flow 24 on the interior of the expandable component 14 is facilitated by a vacuum/suction that is applied to the space 26 between the expandable component 14 and the shaft/hollow tube-like structure 16. This vacuum/suction is less than the force required to expand the expandable component 14 into contact with the tissue to be warmed/cooled, which is the esophagus 6 in this embodiment. As the warming/cooling fluid flows within and around the interior of the expandable component 14, it warms/cools the esophageal tissue 6 adjacent the ablation site 12 thereby reducing the likelihood of damaging the tissue 6 as a result of the ablation.

In some embodiments, flow of the warming/cooling fluid is pulsed or sped up and slowed down. Pulsing the flow of the fluid causes the flexible elements 18 and hence, the temperature sensors 20 included on the free ends and in contact with the expandable component 14, to move back and forth as indicated by arrows 27 in FIG. 2. When the flexible elements 18 and temperature sensors 20 move back and forth in this manner along the interior surface of the expandable component 14, the temperature sensors 20 are able to measure the temperature of a larger area of the tissue in contact with the expandable component 14. This ensures that the temperature of most if not all of the tissue in contact with the expandable component 14 is being measured. This also reduces the number of flexible elements 18 and temperature sensors 20 that are necessary to adequately measure the temperature of the tissue in contact with the expandable component 14 as movement of the flexible elements 18 causes the temperature sensors 20 to measure/monitor a larger tissue area than if they were stationary. In some embodiments, the temperature sensors 20 are used to measure the temperature of the fluid adjacent to the interior wall of the expandable component 14

In some embodiments, the temperature sensors may not contact the interior surface of the expandable component 14 and instead extend only partially into the interior volume of the expandable component 14. In these embodiments, the temperature sensors 20 will be monitoring/measuring the temperature of the fluid flowing on the interior of the expandable component 14. Thus, it is important that the fluid flowing within the expandable component 14 not remain stagnant and must have sufficient flow to ensure adequate thermal transfer between the tissue in contact with the exterior of the expandable component and the fluid flowing on the interior of the expandable component. The speed of the flowing fluid as well as pulsing the flow, helps to ensure adequate mixing and adequate thermal transfer, which results in reducing possible damage to the tissue through better temperature monitoring. Stirring/movement of the fluid within the expandable component 14, is important in detecting temperature changes of the fluid and hence the tissue in contact with the expandable component 14 in order to prevent damage to the tissue.

In some embodiments, the shaft/hollow tube-like structure 16 is connected to a motor, which is used to rotate the shaft/hollow tube-like structure 16. In some embodiments, the motor can be a stepper motor. Rotating the shaft/hollow tube-like structure 16 causes the flexible elements 18 and the temperature sensors 20 to rotate in a corresponding manner. Rotation of the flexible elements 18 and the temperature sensors 20 allows the temperature sensors 20 to measure/monitor the temperature of (1) a larger area of tissue in contact with the expandable component 14 and/or (2) a larger volume of fluid within the expandable component 14. This ensures that the temperature of most if not all of the tissue in contact with the expandable component 14 is being measured and also reduces the number of flexible elements 18 and temperature sensors 20 that are necessary to adequately measure the temperature of the tissue in contact with the expandable component 14 or the volume of fluid within the expandable component 14.

The motor may communicate with a computer that controls the speed of rotation and/or the degree of rotation of the shaft/hollow tube-like structure 16. As will be understood by those of skill in the art, rotation of the shaft/hollow tube-like structure 16 may be achieved with devices other than a stepper motor. In some embodiments, the shaft/hollow tube-like structure 16 can be rotated in combination with pulsing or speeding up and slowing down of the flow of the warming/cooling fluid. Combining rotation with fluid flow pulsing may provide that the temperature of an even greater area of the expandable component 14 can be measured/monitored and can also further reduce the number of flexible elements 18 and temperature sensors 20 that are necessary to measure the temperature of the same amount of tissue in contact with the expandable component 14 than if only rotation or flow pulsing is used. This combination of pulsing and rotating will also provide greater mixing/stirring of the fluid within the expandable component.

Depicted in FIG. 4 is the distal portion of an embodiment of the shaft/hollow tube-like structure 16. In this embodiment, the flexible elements 18 are mounted to the shaft/hollow tube-like structure 16 by way of a mounting collar 28. The mounting collar 28 can be made of the same material as the shaft/hollow tube-like structure 16 or it can be made from a different material. In other embodiments, the flexible elements 18 can be mounted directly to the shaft/hollow tube-like structure 16.

In some embodiments, the shaft/hollow tube-like structure 16 need not be hollow. In these embodiments, supply and return of the warming/cooling fluid to/from the interior of the expandable component 14 can be achieved by fluid supply and fluid return lumens. These fluid supply and fluid return lumens may be located on the exterior of the shaft/hollow tube-like structure 16 or they may be integrated into the walls of the shaft/hollow tube-like structure 16.

In some embodiments, supply of warming/cooling fluid is not included and thus, the shaft/hollow tube-like structure 16 need not be hollow and/or fluid supply and fluid return lumens are not necessary. In these embodiments, the device is used to measure/monitor the temperature of tissue adjacent to the expandable component 14.

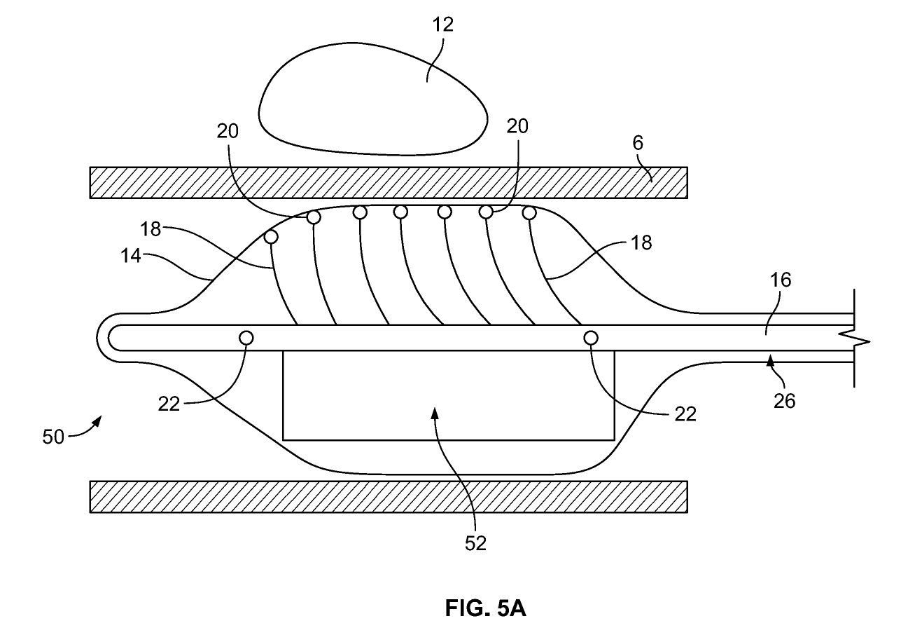

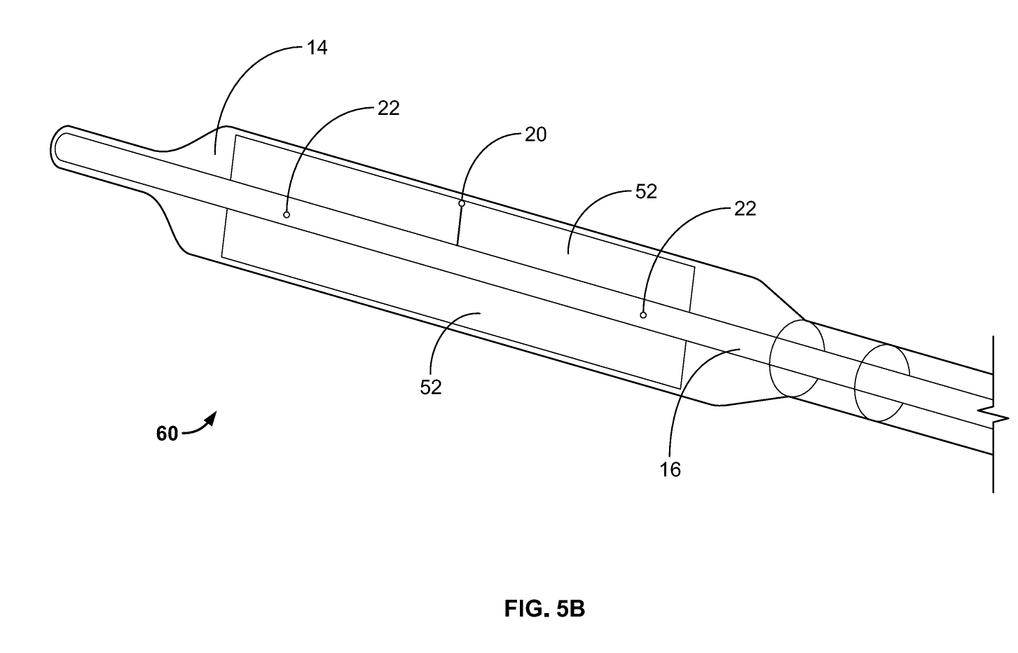

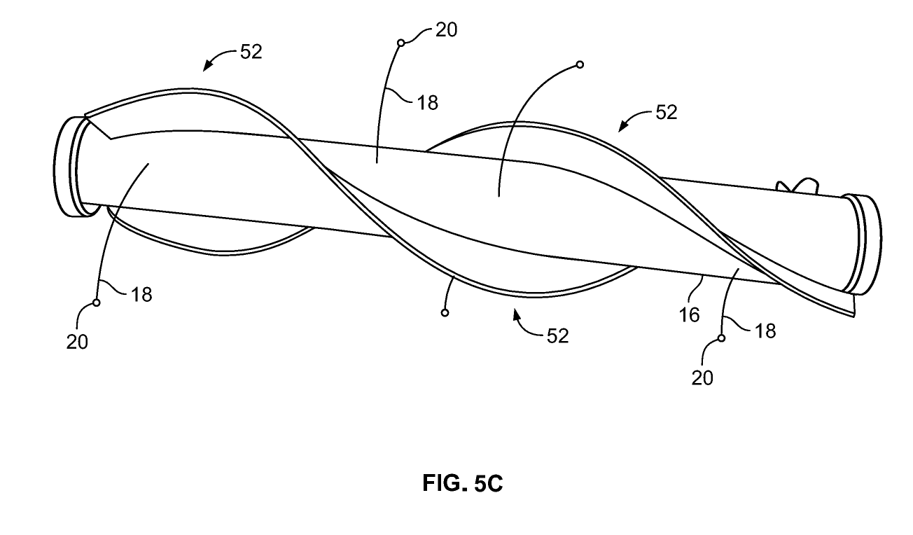

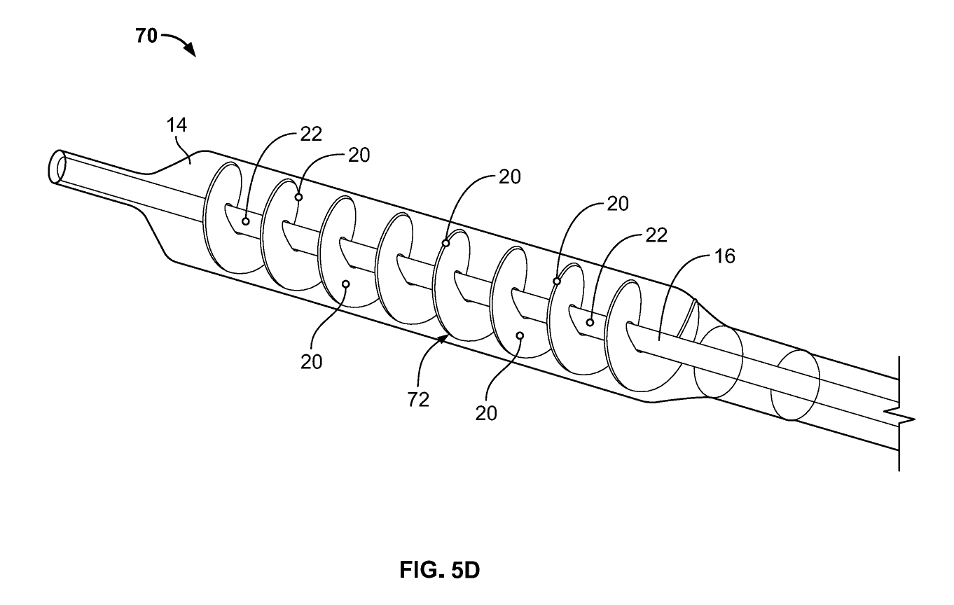

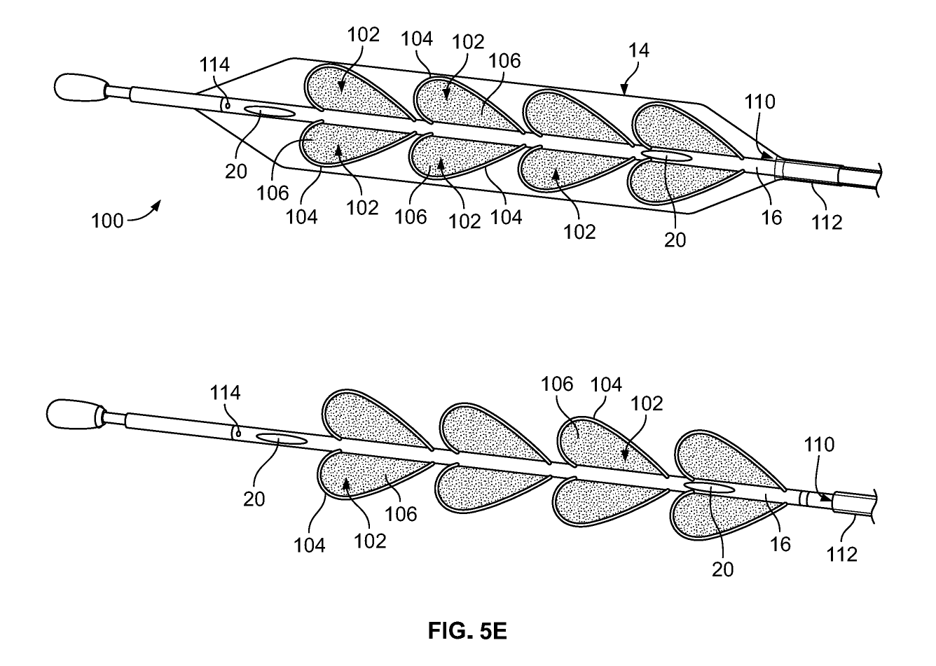

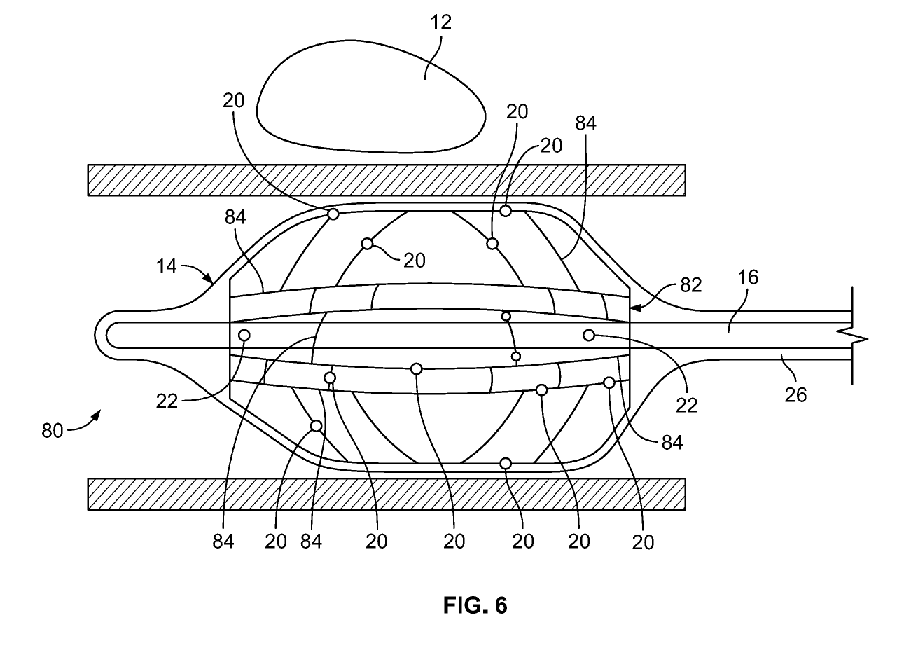

Depicted in FIGS. 5 and 6 are additional embodiments of the device. These embodiments are similar in construction and operation to the previously disclosed embodiments except as set forth below.

In the embodiment depicted in FIG. 5A, the device 50 includes an expandable component 14 such as, for example, a balloon, and a shaft/hollow tube-like structure 16 upon which are mounted a plurality of flexible elements 18, which can be of any type and construction previously disclosed. Included on the free ends of the flexible elements 18 are temperature sensors 20. The shaft/hollow tube-like structure 16 includes at least one fluid delivery port 22, which allows fluid to be delivered from the interior of the shaft/hollow tube-like structure 16 to the interior of the expandable component 14. In some embodiments, 2, 3, 4, 5, 6 or more fluid ports 22 are included. The fluid ports 22 can be used to supply fluid or remove fluid from the interior of the expandable component 14. In this embodiment, the device 50 includes a paddle structure 52. Accordingly, when warming/cooling fluid is delivered to the interior of the expandable component 14 and the shaft/hollow tube-like structure 16 is rotated, the paddle structure 52 causes the warming/cooling fluid to move in a corresponding manner as the rotating paddle structure 52 (1) allowing newly supplied warming/cooling fluid to mix with fluid already contained within the expandable component 14 and/or (2) provide adequate stirring/movement of fluid already contained with the expandable component 14, which, is important in detecting temperature changes of the fluid and hence the tissue in contact with the expandable component 14 in order to prevent damage to the tissue. Inclusion and rotation of the paddle structure 52 also ensures that the warming/cooling fluid adjacent to the ablation site 12 is constantly being moved and replaced allowing for more efficient warming/cooling of tissue adjacent to the ablation site 12. In the depicted embodiment, one paddle structure 52 is included, however, 2, 3, 4, 5 or any number of paddle structures 52 may be included. In some embodiments, temperature sensors 20 can be included on the paddle structure 52. The temperature sensors 20 can be placed at varying lengths away from the shaft/hollow tube-like structure 16 to ensure adequate monitoring of the fluid temperature.

In the embodiment depicted in FIG. 5B the device 60 includes an expandable component 14 such as, for example, a balloon, and a shaft/hollow tube-like structure 16 upon which are mounted two paddle structures 52. Included on at least one of the paddle structures 52 is a temperature sensors 20. In some embodiments, two paddle structures 52 are included. In some embodiments, both paddle structures 52 include at least one temperature sensor 20. In some embodiments, more than two paddle structures 52 are included. The shaft/hollow tube-like structure 16 includes at least one fluid delivery port 22, which allows fluid to be delivered from the interior of the shaft/hollow tube-like structure 16 to the interior of the expandable component 14. In some embodiments, 2, 3, 4, 5, 6 or more fluid ports 22 are included. The fluid ports 22 can be used to supply fluid or remove fluid from the interior of the expandable component 14. Accordingly, when warming/cooling fluid is delivered to the interior of the expandable component 14 and the shaft/hollow tube-like structure 16 is rotated, the paddle structure(s) 52 cause the warming/cooling fluid to move in a corresponding manner as the rotating paddle structure(s) 52 (1) allowing newly supplied warming/cooling fluid to mix with fluid already contained within the expandable component 14 and/or (2) provide adequate stirring/movement of fluid already contained with the expandable component 14, which, is important in detecting temperature changes of the fluid and hence the tissue in contact with the expandable component 14 in order to prevent damage to the tissue. Inclusion and rotation of the paddle structure(s) 52 also ensures that the warming/cooling fluid adjacent to the ablation site 12 is constantly being moved and replaced allowing for more efficient thermal transfer, i.e., warming/cooling, of tissue adjacent to the ablation site 12.

Depicted in FIG. 5C is an embodiment of the shaft/hollow tube-like structure 16 that includes a plurality of flexible elements 18 and two paddle structure 52. In this embodiment, the paddle structure 52 winds around the shaft/hollow tube-like structure 16 in a spiral or corkscrew pattern. Although two paddle structures are shown, 1, 3, 4, 5 or any number of paddle structures 52 may be included. Configuring the paddle structures 52 in a spiral or corkscrew pattern around the shaft/hollow tube-like structure 16 causes (1) greater movement/stirring/mixing of the warming/cooling fluid as the shaft/hollow tube-like structure 16 rotates, which is important for the reasons previously discussed and (2) greater movement of the flexible elements 18 thereby allowing the temperature sensors 20 to monitor the temperature of a greater surface area of the expandable component 14. In some embodiments, the paddle structures 52 can be a polymer sheet material, for example.

Depicted in FIG. 5D is an embodiment of the shaft/hollow tube-like structure 16 that includes a single paddle structure 52. In this embodiment, the paddle structure 52 winds around the shaft/hollow tube-like structure 16 in a spiral or corkscrew pattern. Also included is at least one temperature sensor 20 on the paddle structure 52. Although one paddle structure and one temperature sensor are shown, 2, 3, 4, 5 or any number of paddle structures 52 and temperature sensors 20, may be included. Configuring the paddle structure 52 in a spiral or corkscrew pattern around the shaft/hollow tube-like structure 16 causes greater movement/stirring/mixing of the warming/cooling fluid as the shaft/hollow tube-like structure 16 rotates, which is important for the reasons previously discussed. In some embodiments, the paddle structure 52 can be a polymer sheet material, for example.

In the embodiment depicted in FIG. 5E, the device 100 includes an expandable component 14 such as, for example, a balloon, and a shaft/hollow tube-like structure 16 upon which is mounted at least one paddle structure 102 and preferably, a plurality of paddle structures 102. In some embodiments, included on at least one of the paddle structures 102 is a temperature sensor 20. In some embodiments, temperature sensors 20 are also included on the shaft/hollow tube-like structure 16. In some embodiments, as depicted in FIG. 5E, one or more temperature sensors 20 are only included on the shaft/hollow tube-like structure 16 and not on the paddle structures 102. In some embodiments, 1, 2, 3, 4, 5, 6, 7, 8, 9, 10 or more paddle structures 102 are included.

In some embodiments, the paddle structures 102 include (a) a flexible/collapsible frame 104, which can be made of any flexible/collapsible material such as, for example, nitinol and (b) a film component/material 106 such as, for example, a plastic material, that covers the frame 104 and which facilitates stirring/mixing/movement of the fluid on the interior of the expandable component 14 as the shaft structure 16 rotates. In some embodiments, the film material 106 is fused to the frame 104. Such a construction allows the paddle structures 102 to collapse down so that the diameter of the device 100 can be minimized during delivery to the desired body lumen/cavity through, for example, a delivery catheter. As will be understood by those of skill in the art, the frame 104 can be any material that, (i) can be collapsed down during delivery of the device 100 and (ii) provides sufficient structure to the attached film material 106 in an un-collapsed state such that when the shaft structure 16 and hence the paddle structure 102 are rotated, the paddle structure 102 is capable of stirring/mixing or promoting movement of the fluid on the interior of the expandable component 14. As will be understood by those of skill in the art, the film material 106 can be any material that, (i) can be collapsed down during delivery of the device 100 and (ii) when attached to the frame 104 and the shaft structure 16 and hence the paddle structure 102 are rotated, is capable of stirring/mixing or promoting movement of the fluid on the interior of the expandable component 14.

In some embodiments, fluid is delivered to the interior of the expandable component 14 through either (i) a fluid port 22 in the shaft/hollow tube-like structure 16 or (ii) an annular space 110 between the shaft/hollow tube-like structure 16 and a second tube-like structure 112 that surrounds the shaft/hollow tube-like structure 16 as depicted in FIG. 5E. In order to allow fluid to be removed or to allow fluid to circulate through the interior of the expandable component 14, a fluid outflow port 114 is included on the shaft/hollow tube-like structure 16.

Accordingly, in some embodiments, when warming/cooling fluid is delivered to the interior of the expandable component 14 and the shaft/hollow tube-like structure 16 is rotated, the paddle structure(s) 102 cause the warming/cooling fluid to move in a corresponding manner as the rotating paddle structure(s) 102 (1) allowing newly supplied warming/cooling fluid to mix with fluid already contained within the expandable component 14 and/or (2) provide adequate stirring/movement of fluid already contained with the expandable component 14, which is important in detecting temperature changes of the fluid and hence the tissue in contact with the expandable component 14 in order to prevent damage to the tissue. Inclusion and rotation of the paddle structure(s) 102 also ensures that the warming/cooling fluid adjacent to the ablation site 12 is constantly being moved and replaced allowing for more efficient thermal transfer, i.e., warming/cooling, of tissue adjacent to the ablation site 12 thereby further reducing the potential of damaging/ablating tissue adjacent to the ablation site.

It is important to note that in all of the embodiments disclosed and described herein, adequate stirring/mixing/movement of the fluid on the interior of the expandable component 14 is very important in providing a homogenous fluid temperature in order to ensure that the temperature sensors 20 adequately and efficiently monitor/measure and identify any temperature changes to the fluid within the interior of the expandable component 14. Thus, monitoring the temperature of the fluid volume ensures that this device and method will detect a temperature change within the entire fluid volume rather than a temperature change of a discreet location within the flexible structure, which may be more challenging and which may not sufficiently monitor the temperature of all of the tissue in contact with the expandable component, i.e., locations of the interior of the expandable component 14 may not be sufficiently monitored resulting in temperature changes that may go undetected.

In the embodiment depicted in FIG. 6, the device 80 includes an expandable component 14 such as, for example, a balloon, and a shaft/hollow tube-like structure 16 upon which is mounted a stent structure 82. The stent structure 82 includes a plurality of struts 84. Mounted on the struts 84 are a plurality of temperature sensors 20. The temperature sensors 20 can be mounted on as many of the struts 84 as is necessary to obtain the number of temperature measurements desired. Additionally, multiple temperatures sensors 20 can be mounted on the same strut 84. Temperature sensors 20 can be placed on portions of the struts 84 so that they contact the interior surface of the expandable component 15 when the stent structure 82 is expanded such that the temperature sensors 20 monitor the temperature of the tissue in contact with the expandable component 14. Temperature sensors 20 can also be placed on portions of the struts 84 so they are located at different locations within the interior volume of the expandable component 14 such that the fluid temperature on the interior of the expandable component 14 can be monitored at different areas within the volume on the interior of the expandable component, away from the expandable component's interior surface.

Stent structure 82 can be made from any shape memory alloy such as, for example, nitinol such that when the stent structure 82 is delivered to the target area within the expandable component 14 adjacent to the ablation site 12, it expands thereby also expanding the expandable component 14 into contact with the body tissue 6. In some embodiments, the stent structure 82 is balloon expandable and includes a balloon on its interior in order to expand the stent structure 82 and the expandable component 14 into contact with the body tissue 6. Once expanded, the expansion balloon is removed from the device. The shaft/hollow tube-like structure 16 includes at least one fluid delivery port 22, which allows fluid to be delivered from the interior of the shaft/hollow tube-like structure 16 to the interior of the expandable component 14.

In all of the disclosed embodiments of the device 10, 50, 80, a processing device such as, for example, a computer is used to (1) connect to the temperature sensors 20 to record the temperature measurements and/or (2) to control the warming/cooling fluid flow and temperature. The computer can be programmed such that if a temperature sensor 20 measures a temperature value(s) that indicate the tissue temperature has been altered by the ablation procedure, the physician performing the ablation procedure is alerted. The computer can also use algorithms to analyze temperature measurements using probability and statistics or any other methods used to interpret data measurements. In some embodiments, the computer either is the same computer that controls the ablation procedure or communicates with the computer that controls the ablation procedure such that if a temperature sensor 20 measures a temperature and the algorithm interprets the temperature measurements such that tissue damage is possible if ablation treatment continues, the computer automatically halts the ablation procedure thereby preventing possible damage to the tissue. Additionally, the computer can be programmed such that if the temperatures measured by the temperature sensors 20 begin to approach a value nearing possible tissue damage, the computer can (1) increase flow of the warming/cooling fluid and/or (2) increase speed of the motor to increase rotation and hence mixing/movement of the fluid within the expandable component 14, in order to increase the warming/cooling power of the device in order to prevent possible damage to the tissue 6.

In use, the device is delivered to the site of interest within a body lumen using any know delivery means such as, for example, a delivery catheter. Once at the site and prior to commencing the ablation procedure, the expandable member 14 is expanded. When the expandable member 14 is a balloon, the balloon is inflated either with an inflation fluid or by commencing the flow of the warming/cooling fluid. If the device is just being used to measure/monitor tissue temperature, warming/cooling fluid is not used to expand the expandable member 14 into contact with the lumen tissue. In some embodiments, the expandable member 14 is delivered separately from the shaft/hollow tube-like structure 16. In these embodiments, once the expandable member 14 is delivered to the point of interest within the lumen, the shaft/hollow tube-like structure 16 is then delivered to the interior of the expandable member 14.

After or simultaneously with the expansion/inflation of the expandable member 14, the flexible elements 18 also expand/spring open up such that the temperature sensors 20 on the free ends of the flexible elements 18 contact the interior surface of the expandable member 14. Similarly, in embodiments where the device includes temperature sensors 20 on a stent structure 82, the stent structure 82 is expanded either as a result of shape memory alloys used for its construction or with the use of a balloon, to ensure that the struts 84 and hence any temperature sensors 20 thereon, contact the interior of the expandable member 14. After the expandable member 14 is expanded/inflated and the associated temperature sensors 20 of the device are moved into contact with the expandable member's interior surface, the device can now be used to measure the temperature of the lumen tissue in contact with the exterior surface of the expandable member 14.

If the device is used to also warm/cool the lumen tissue adjacent the ablation site 12, flow of the warming/cooling fluid is commenced. As previously disclosed, the warming/cooling fluid is delivered to the interior of the expandable member 14 through a lumen of the shaft/hollow tube-like structure 16. At the distal end of the device, the warming/cooling fluid exits the fluid ports 22 and enters the interior of the expandable member 14. The warming/cooling fluid flows around the interior of the expandable component 14 (depicted by arrows 24 in FIG. 2), contacting the interior surface of the expandable component 14 thereby warming or cooling the tissue in contact with the exterior surface of the expandable component 14. The cooled/heated fluid is then removed from the interior of the expandable component 14 through the space 26 between the expandable component 14 and the shaft/hollow tube-like structure 16. Depending on the embodiment of the device, in order to facilitate more comprehensive measuring/monitoring of the tissue temperature by the temperature sensors 20 and/or to facilitate more effective warming/cooling fluid flow/missing/stirring within the interior of the expandable component 14, the fluid flow is pulsed and/or the shaft/hollow tube-like structure 16 is rotated.

As previously discussed, in some embodiments, if any of the temperature sensors 20 measure a temperature that is approaching a limit that is determined by the computer with the use of algorithms, for example, either (1) the warming/cooling fluid flow can be increased and/or (2) the physician is alerted to this condition and/or (3) the ablation procedure is halted.

In order to remove the device, the expandable component 14 is collapsed/deflated, which also causes the flexible elements 18 or any stent structure to collapse down as well. Thus, the entire device can be removed from the area of interest within the body through, for example, a catheter.

Many modifications and variations of the present invention are possible in light of the above teachings. It is therefore to be understood that within the scope of the appended claims the invention may be practiced otherwise than as specifically described.

1.-41. (canceled)

42. A device for monitoring temperature adjacent to a body lumen comprising:
an expandable component; and
a shaft comprising:
a lumen;
at least one fluid port in communication with the lumen;
at least one paddle structure connected to the shaft; and
at least one temperature sensor mounted thereon.

43. The device of claim 42, wherein the paddle structure comprises a frame structure and a film component

44. The device of claim 42, further comprising a motor to rotate the shaft.

45. The device of claim 42, wherein the temperature sensor is selected from the group comprising thermocouples, Resistance Temperature Detectors (RTDs) and thermistors.

46. The device of claim 42, further comprising a plurality of paddle structures.

47. The device of claim 43, wherein the frame structure comprises a flexible material.

48. The device of claim 43, wherein the frame structure comprises nitinol.

49. The device of claim 43, wherein the film component is a flexible material.

50. The device of claim 43, wherein the flexible material is a plastic material.

51. A device for monitoring temperature adjacent to a body lumen comprising:
an expandable component;
a first shaft comprising:
a lumen;
at least one fluid port in communication with the lumen;
at least one paddle structure connected to the shaft; and
a second shaft surrounding at least a portion of the first shaft and having a gap formed between the first shaft and the second shaft, wherein the gap is for supplying a fluid to an interior of the expandable component; and
at least one temperature sensor.

52. The device of claim 51, wherein the at least one paddle structure comprises a frame structure and a film component.

53. The device of claim 51, wherein the at least one temperature sensor is mounted the first shaft.

54. The device of claim 51, further comprising a motor to rotate the shaft.

55. The device of claim 51, wherein the temperature sensor is selected from the group comprising thermocouples, Resistance Temperature Detectors (RTDs) and thermistors.

56. The device of claim 52, wherein the frame structure and the film component comprises a flexible material.

57. The device of claim 51, wherein the expandable component is a balloon.

58. A device for monitoring temperature adjacent to a body lumen comprising:
a balloon; and
a shaft on an interior of the balloon, the shaft comprising:
a lumen;
at least one fluid port in communication with the lumen;
a plurality of flexible paddle structures connected to the shaft, wherein each paddle structure comprises (i) a frame structure and (ii) a film component attached to the frame structure; and
at least one temperature sensor mounted on the shaft.

59. A method of monitoring tissue temperature comprising:
delivering a tissue temperature monitoring device to a location within the human body adjacent to a site of interest, the tissue temperature monitoring device comprising:
an expandable component; and
a shaft comprising:
a lumen;
at least one fluid port in communication with the lumen; and
at least one paddle structure connected to the shaft and comprising (i) a frame structure and (ii) a film component; and
at least one temperature sensor mounted on the shaft;
filling the expandable component with a fluid;
rotating the shaft; and
monitoring a temperature of the fluid within the expandable component with the at least one temperature sensor.

60. The method of claim 59, wherein the fluid is a warming fluid.

61. The method of claim 59, further comprising a plurality of paddle structures.
